(19) United States
(12) Patent Application Publication
(10) Pub. No.: US 2026/0364258 A1
(43) Pub. Date: Nov. 19, 2026

(54) INJECTION SYSTEMS AND METHODS

(71) Applicant: CREDENCE MEDSYSTEMS, INC., Menlo Park, CA (US)

(72) Inventors: Everett Matthew Notaro, Santa Clara, CA (US); Alan E. Shluzas, San Carlos, CA (US)

(73) Assignee: CREDENCE MEDSYSTEMS, INC., Menlo Park, CA (US)

(21) Appl. No.: 19/410,426

(22) Filed: Dec. 5, 2025

Related U.S. Application Data
(60) Provisional application No. 63/728,412, filed on Dec. 5, 2024.

(51) Int. Cl. A61M 5/20; A61M 5/31; A61M 5/315; A61M 5/32; A61M 5/46; A61M 5/50

(52) U.S. Cl. CPC A61M 5/20; A61M 5/3147; A61M 5/31566; A61M 5/3234; A61M 5/46; A61M 5/502; A61M 2005/3241; A61M 2205/58

(57) ABSTRACT

An injection system includes a syringe body, a needle hub assembly, a stopper member, and a plunger member. The needle hub assembly includes a needle hub, a needle removably coupled to the needle hub, and a compression ring disposed around a proximal end of the needle hub and configured to couple the needle hub to the syringe body distal end. The plunger member has a plunger member proximal end and a plunger member distal end, and coupled to the stopper member at the plunger member distal end. The plunger member is configured to insert the stopper member distally in the syringe interior relative to the syringe body and the needle hub. The injection system also includes a shroud coupled to the syringe flange. The shroud is configured to couple to the syringe flange within one or more ranges of tolerance for the syringe body.

The present application claims priority to U.S. Provisional Patent Application Ser. No. 63/728,412, filed on Dec. 5, 2024 under attorney docket number CM.30048.00 and entitled “INJECTION SYSTEMS AND METHODS.” This application includes subject matter similar to the subject matter described in the following co-owned U.S. patent applications: (1) U.S. patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”; (16) U.S. Provisional Patent Application Ser. No. 63/537,641, filed on Sep. 11, 2023 under attorney docket number CM.30037.01 and entitled “SAFE INJECTION SYSTEMS AND METHODS”; and (16) U.S. patent application Ser. No. 18/946,512, filed on Nov. 13, 2024 under attorney docket number CM.20044.00 and entitled “SAFE INJECTION SYSTEMS AND METHODS”. The contents of the applications and patents identified herein are fully incorporated herein by reference as though set forth in full.

### FIELD OF THE DISCLOSURE

The present disclosure relates generally to injection systems, devices, and processes for facilitating various levels of control over injection processes, and more particularly to injection systems with housings, syringes for use with same, and methods related to injections using such injection systems and syringes, with or without safety features, in healthcare environments.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus, certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Some injection systems such as those described in U.S. Pat. Nos. 10,010,677 and 10,926,038, which were previously incorporated by reference herein, include an autoinjector or a pen injector, and a syringe for use therein. However, acceptable tolerances in various dimensions of syringes complicate interfaces between autoinjectors/pen injectors and syringes. Accordingly, there exists a need for safe injection systems with autoinjectors/pen injectors and syringes that address these limitations.

### SUMMARY

Embodiments are directed to injection systems. In particular, the embodiments are directed to safe injection systems.

According to one embodiment, an injection system includes a syringe body. The syringe body includes a syringe body proximal end, a syringe body distal end, and a syringe body interior therebetween. The syringe body also includes a syringe flange at the syringe body proximal end. The injection system also includes a needle hub assembly coupled to the syringe body at the syringe body distal end. The needle hub assembly includes a needle hub and a needle removably coupled to the needle hub. The needle has a sharp needle distal end and a needle proximal end feature. The needle hub assembly also includes a compression ring disposed around a proximal end of the needle hub and configured to couple the needle hub to the syringe body distal end. The injection system further includes a stopper member movably disposed in the syringe interior. Moreover, the injection system includes a plunger member having a plunger member proximal end and a plunger member distal end, and coupled to the stopper member at the plunger member distal end. The plunger member is configured to insert the stopper member distally in the syringe interior relative to the syringe body and the needle hub. The injection system also includes a shroud coupled to the syringe flange. The shroud is configured to couple to the syringe flange within one or more ranges of tolerance for the syringe body.

In one or more embodiments, the one or more ranges of tolerance includes a range of tolerance for a length of the syringe body. The range of tolerance for the length of the syringe body may be −1.5 mm to +1 mm. The one or more ranges of tolerance may include a range of tolerance for a diameter of the syringe body. The range of tolerance for the diameter of the syringe body may be −0.5 mm to +0.5 mm. The one or more ranges of tolerance may include a range of tolerance for a diameter of the syringe flange. The range of tolerance for the diameter of the syringe flange may be −0.75 mm to +0.75 mm.

In one or more embodiments, the compression ring is configured to function as a registration flange for an autoinjector. The shroud may be configured to function as a registration flange for an autoinjector. The syringe body interior may be configured to hold 1 ml of liquid. The syringe body interior may be configured to hold 2.5 ml of liquid. The injection system may include a needle retraction system configured to retract the sharp needle distal end into the needle hub to render the injection system safe.

According to another embodiment, an injection system includes an autoinjector and a syringe. The autoinjector includes a housing. The syringe includes a syringe body having a syringe body proximal end, a syringe body distal end, and a syringe body interior therebetween. The syringe also includes a syringe flange at the syringe body proximal end. The injection system also includes a needle hub assembly coupled to the syringe body at the syringe body distal end. The needle hub assembly includes a needle hub and a needle removably coupled to the needle hub. The needle has a sharp needle distal end and a needle proximal end feature. The needle hub assembly also includes a compression ring disposed around a proximal end of the needle hub and configured to couple the needle hub to the syringe body distal end. The injection system further includes a stopper member movably disposed in the syringe interior. Moreover, the injection system includes a plunger member having a plunger member proximal end and a plunger member distal end, and coupled to the stopper member at the plunger member distal end. The plunger member is configured to insert the stopper member distally in the syringe interior relative to the syringe body and the needle hub. In addition, the injection system includes a shroud coupled to the syringe flange. The shroud is configured to couple to the syringe flange within one or more ranges of tolerance for the syringe body.

In one or more embodiments, the one or more ranges of tolerance includes a range of tolerance for a length of the syringe body. The range of tolerance for the length of the syringe body may be −1.5 mm to +1 mm. The one or more ranges of tolerance may include a range of tolerance for a diameter of the syringe body. The range of tolerance for the diameter of the syringe body may be −0.5 mm to +0.5 mm. The one or more ranges of tolerance may include a range of tolerance for a diameter of the syringe flange. The range of tolerance for the diameter of the syringe flange may be −0.75 mm to +0.75 mm.

In one or more embodiments, the compression ring is configured to function as a registration flange for an autoinjector. The autoinjector may include a distal syringe holder operatively coupled to the compression ring. The shroud may be configured to function as a registration flange for an autoinjector. The autoinjector may include a proximal syringe holder operatively coupled to the compression ring. The syringe body interior may be configured to hold 1 ml of liquid. The syringe body interior may be configured to hold 2.5 ml of liquid. The injection system may include a needle retraction system configured to retract the sharp needle distal end into the needle hub to render the syringe safe.

In one or more embodiments, the one or more ranges of tolerance of the shroud for the syringe body includes a length tolerance configured to maintain a predetermined depth of the needle to be inserted into a patient by the autoinjector. The plunger member may include a proximal interface as the plunger member proximal end. In a safe configuration after injection, the proximal interface may be disposed in an interior of the shroud with a proximal surface of the proximal interface disposed distal of a proximal end surface of the shroud.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

FIGS. 1A to 5C illustrate various aspects of conventional injection syringe configurations.

FIGS. 6 and 7 are perspective and longitudinal cross-sectional views of a safe injection system in a ready-to-use configuration according to some embodiments.

FIGS. 8 and 9 are back and front perspective views of a syringe body and needle hub assembly of an injection system with a shroud in position for assembly according to some embodiments.

FIGS. 10 and 11 are back perspective and cutaway back perspective views of a shroud for use with a syringe body of an injection system according to some embodiments.

FIGS. 12, 13, and 14 are front, side, and back views of a shroud for use with a syringe body of an injection system according to some embodiments.

FIGS. 15 and 16 are perspective and longitudinal cross-sectional views of a safe injection system in a safe configuration according to some embodiments.

FIG. 17 is a schematic view of a safe injection system disposed in a two-part injector having an external housing in a ready-to-use configuration according to some embodiments.

FIGS. 18 and 19 are perspective and longitudinal cross-sectional views of a safe injection system in a ready-to-use configuration according to some embodiments.

FIGS. 20 and 21 are back and front perspective views of a syringe body and needle hub assembly of an injection system with a shroud in position for assembly according to some embodiments.

FIGS. 22 and 23 are back perspective and cutaway back perspective views of a shroud for use with a syringe body of an injection system according to some embodiments.

FIGS. 24, 25, and 26 are front, side, and back views of a shroud for use with a syringe body of an injection system according to some embodiments.

FIGS. 27 and 28 are perspective and longitudinal cross-sectional views of a safe injection system in a safe configuration according to some embodiments.

FIG. 29 is a schematic view of a safe injection system disposed in a two-part injector having an external housing in a ready-to-use configuration according to some embodiments.

FIG. 30 is a schematic view of two safe injection systems configured to be disposed in a two-part injector having an external housing according to some embodiments.

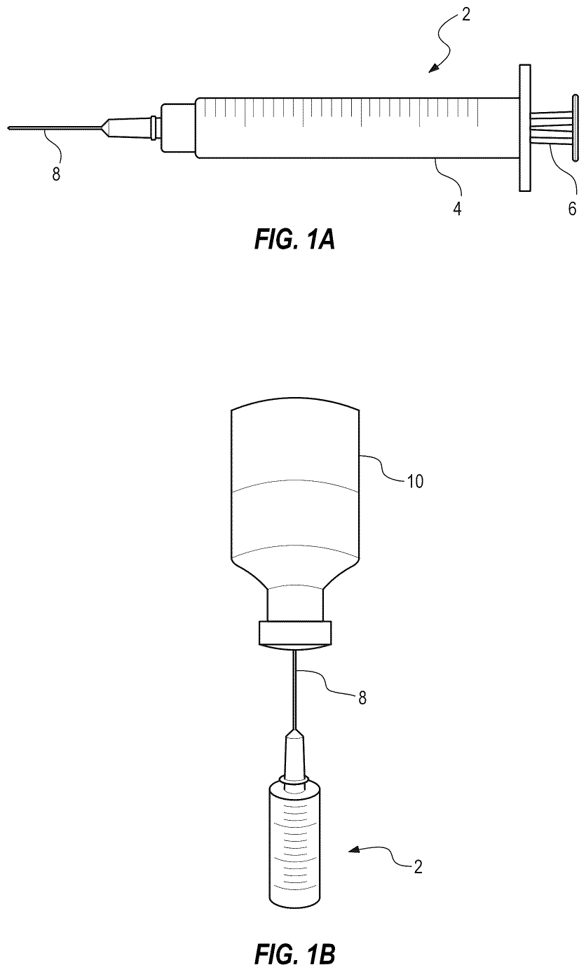

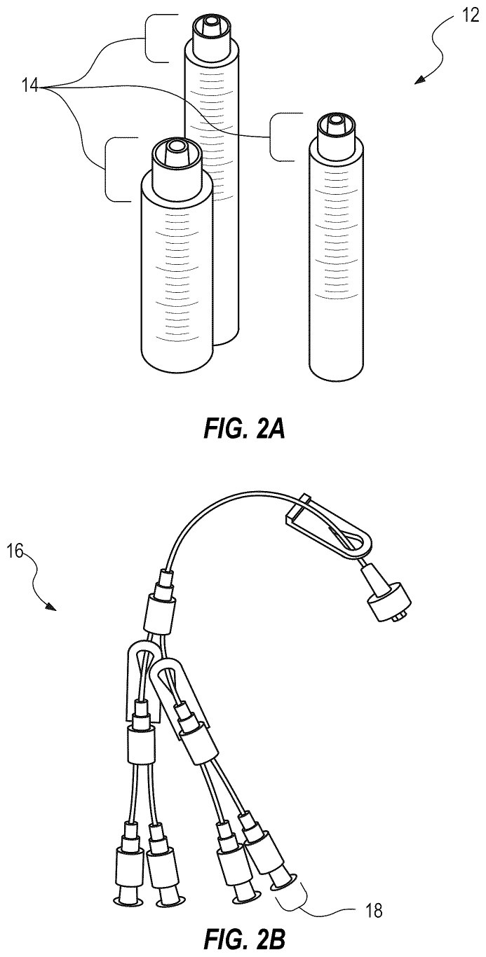

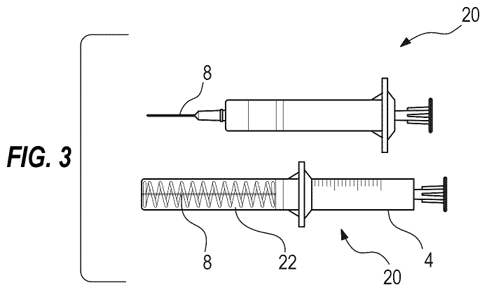

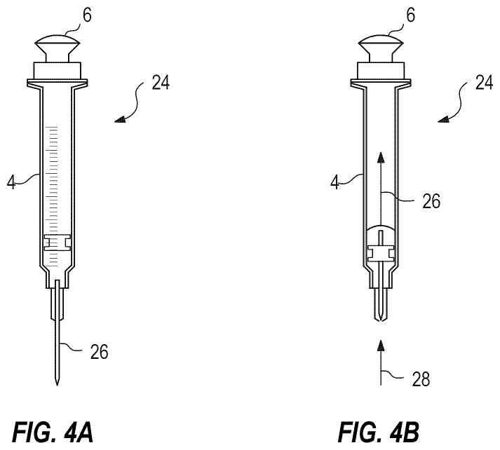

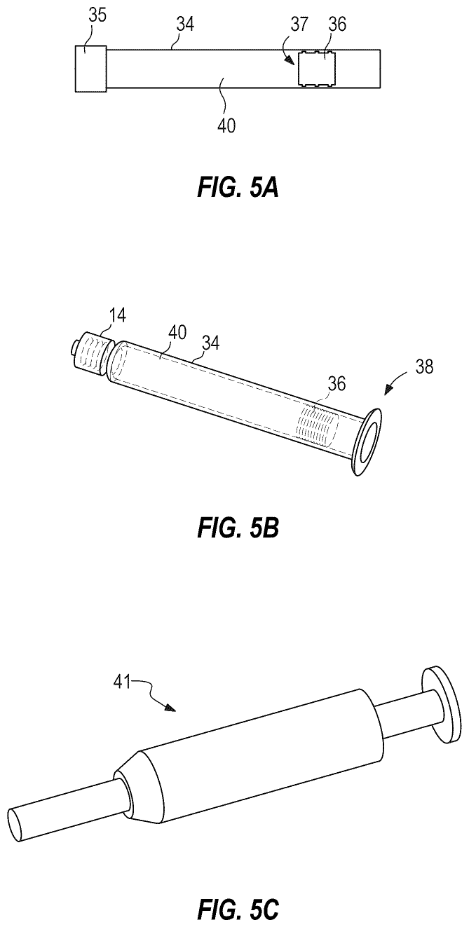

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Safe Injection Systems

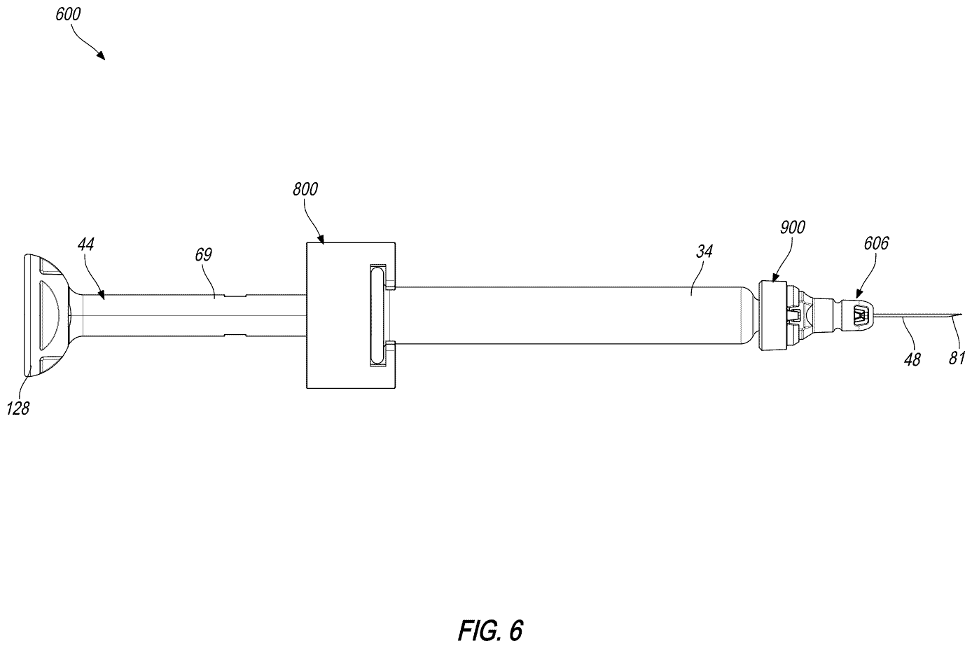

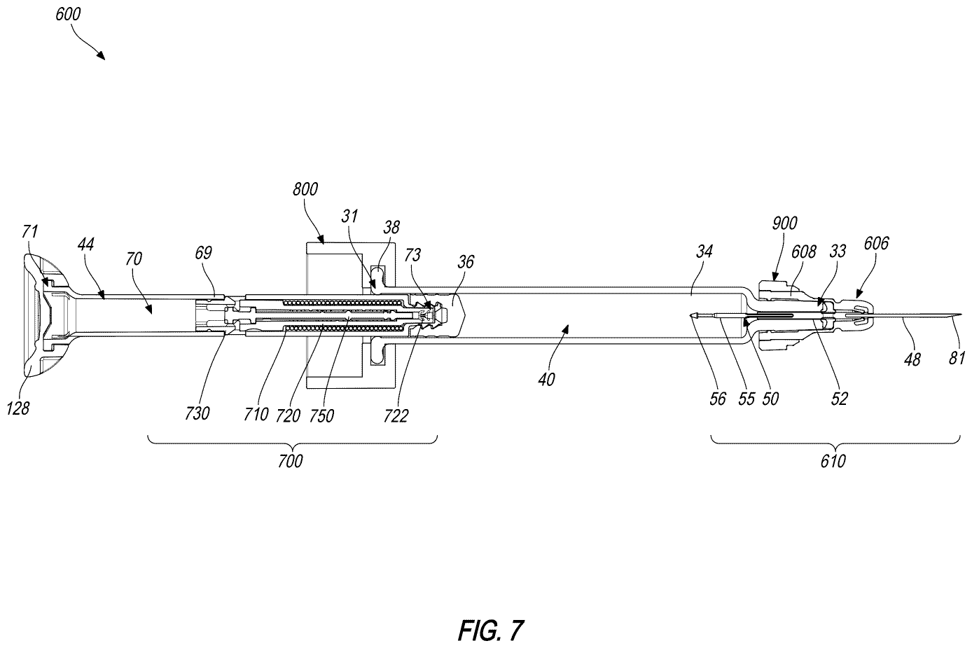

FIGS. 6 and 7 are side and longitudinal cross-sectional views of a safe injection system (600) in a ready-to-use configuration before injection, with a conventional off-the-shelf pre-filled syringe body (34) with a conventional off-the-shelf stopper member (36) disposed therein according to some embodiments. Prior to the ready-to-use configuration, the syringe body (34) is pre-filled with medicine for storage and shipment to the user and/or patient. The syringe body (34) is sealed at a distal end (33) thereof with a rubber and/or polymer rigid needle shield (not shown), which surrounds and seals the distal needle member (48), and sealed at a proximal end (31) thereof with the stopper member (36) to contain the medicine inside the syringe body (34). The syringe body (34) has a syringe body proximal end (31), a syringe body distal end (33), and a radially outward extending syringe flange (38) as the syringe body proximal end (31). The syringe body (34) is configured to be prefilled with a volume of medicine between approximately 0.2 ml to approximately 10 ml of liquid. The illustrative embodiments shown depict syringe bodies sized to deliver volumes of 1 ml, 2.25 ml, and/or 3 ml of medicine. The stopper member (36) together with the syringe body (34) define a syringe chamber (40). The stopper member (36) occludes the proximal end (31) of the syringe chamber (40). The safe injection system (600) also includes a plunger member (44) coupled to a proximal end of the stopper member (36), for instance with a conventional screw-on connection. The plunger member (44) defines a plunger member proximal end (71) and a plunger member distal end (73). A needle hub assembly (606) is disposed at the distal end (33) of the syringe chamber (40).

The safe injection system (600) includes a needle retraction system (700) for use with safe injection systems such as the system (600) depicted in FIG. 7 according to some embodiments. The needle retraction system (700) includes an energy-storage member/spring (710) shown in an energized/compressed state, a needle catch (720), and a trigger (750). Most of the components of the needle retraction system (700) are disposed in the plunger interior (70) defined by the plunger body (69). As such, the plunger member (44) has approximately the same dimensions as a conventional plunger member and is usable with the conventional syringe body (34) and conventional stopper member (36).

The needle catch (720) defines a pair of rotatable energy-storage member latches (730) configured to interfere with the plunger body (69) to hold the energy-storage member (710) in the energized state depicted in FIG. 7. The needle catch (720) also defines a pair of rotatable needle latch arms (722) configured to interfere with the radially expanded member (56) on the needle proximal member (50) to allow the radially expanded member (56) to move proximally past the rotatable needle latch arms (722) while preventing the radially expanded member (56) from moving distally past the plurality of rotatable needle latch arms (722). As such, the needle latch arms (722) allow the radially expanded member (56) and the needle proximal member (50) to be caught or captured by the needle catch (720).

The safe injection system (600) controls injection of a fluid from the syringe chamber (40) through the needle hub assembly (606) driven by distal insertion of the plunger member (44) relative to the syringe body (34). This distal insertion of the plunger member (44) relative to the syringe body (34) may be performed automatically by an autoinjector or manually by a user. The plunger member (44) includes a plunger housing member (69) defining a plunger interior (70) and a proximal interface (128) coupled to the plunger housing member (69) at a proximal end (31) thereof.

The safe injection system (600) has a staked needle configuration wherein upon presentation to the user, a needle hub assembly (606), comprising a needle hub (608) and a needle assembly (610), is mounted in position ready for injection. The distal end (33) of the syringe body (34) comprises a luer taper interface configured to facilitate coupling of the needle hub assembly (606) to the syringe body (34).

The needle assembly (610) includes a needle distal member (48) and a needle proximal member (50) coupled to each other by a needle joining member (52). The needle distal member (48) defines a sharp needle distal end (81). The needle proximal member (50) defines a radially expanded member (56) at a proximal end thereof and a cylindrical portion (55). The needle assembly (610) form a fluid path from the syringe chamber (40), through the needle joining member (52), through the needle distal member (48), and to an exterior of the safe injection system (600).

While the staked needle is depicted as mounted in position, the staked needle may be removably coupled to the syringe body (34) using a Luer interface (not shown), with the proximal member (50) of the needle member extending through the Luer interface and into the syringe chamber (40). Alternatively, the needle may be fixedly or removably mounted to the flange on a cartridge body instead of a syringe. Such cartridge injection systems are disclosed in U.S. patent application Ser. No. 15/801,281, which was previously incorporated by reference herein. Alternatively, the needle may be fixedly or removably mounted to the tip of a dual chamber injection system. Such dual chamber injection systems are disclosed in U.S. patent application Ser. No. 15/801,259, which was previously incorporated by reference herein. Alternatively, the needle may be fixedly or removably mounted to the tip of a sequential injection system. Such dual chamber injection systems are disclosed in U.S. patent application Ser. No. 17/364,546, which was previously incorporated by reference herein.

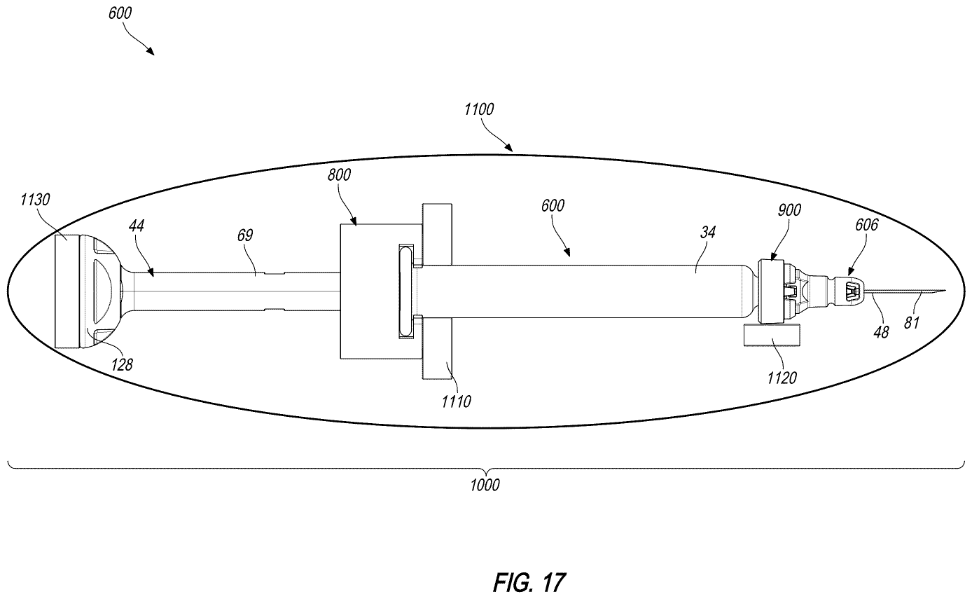

The safe injection system (600) includes a shroud (800) coupled to the syringe flange (38) within ranges of tolerance for the dimensions of the syringe body (34) and the syringe flange (38). The safe injection system (600) also includes a compression ring (900) disposed around a proximal end of the needle hub (608) to couple the needle hub assembly (606) to the distal end (33). The shroud (800) and the compression ring (900) are configured to facilitate use of the safe injection system (600) in autoinjectors, pen injectors, and other two-part injectors having an external housing. The shroud (800) and the compression ring (900) are configured to register with corresponding components of these external housings (see FIG. 17).

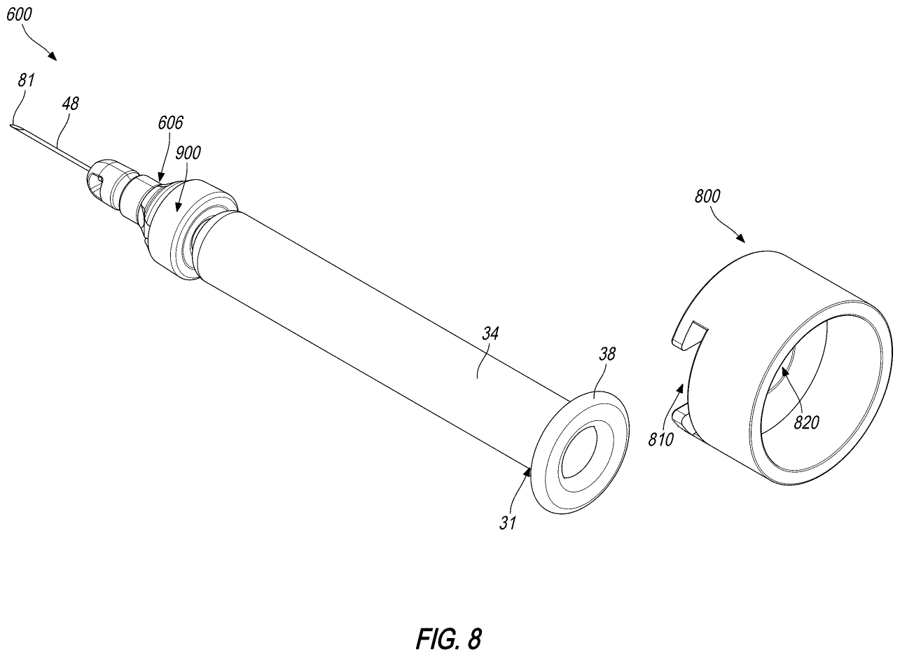

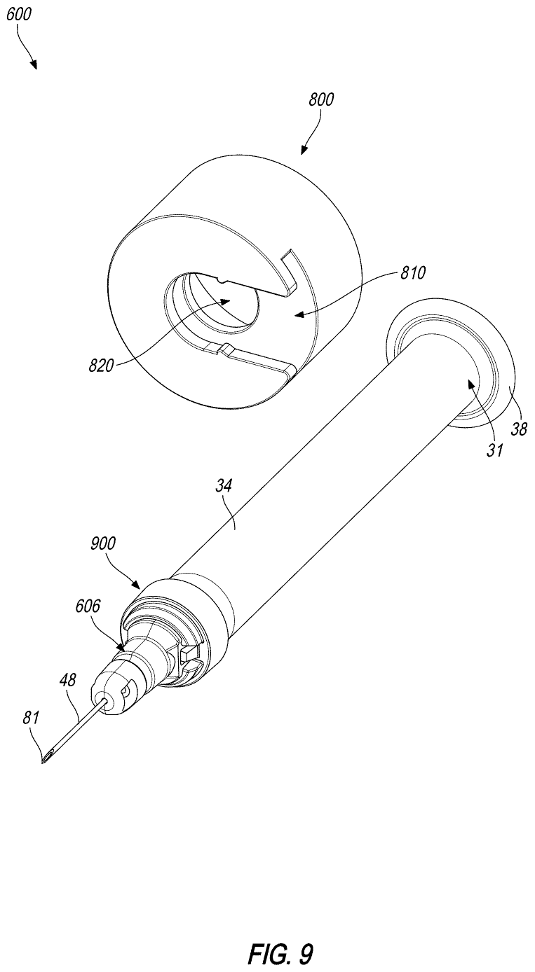

FIGS. 8 and 9 are back and front perspective views of a syringe body (34) and needle hub assembly (606) of an injection system (600) with a shroud (800) in position for assembly according to some embodiments. The shroud (800) defines a side-opening slot (810) configured to allow the shroud to be over the syringe flange (38) to couple the shroud (800) to the syringe flange (38). The shroud also defines a central opening (820) along a longitudinal axis thereof configured to allow the plunger member (44) to be inserted distally through the shroud and into the syringe chamber (40) after the shroud (800) is assembled onto the syringe body (34).

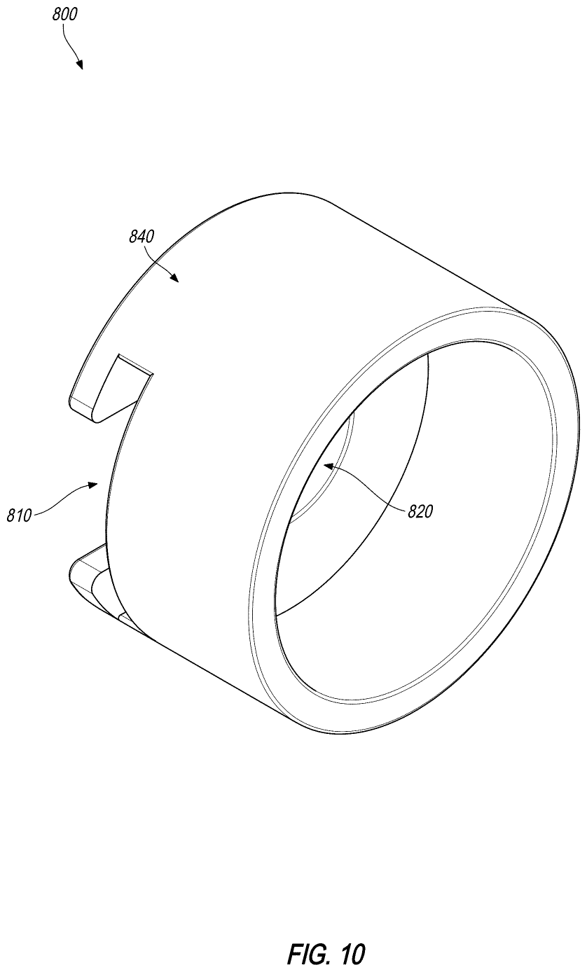

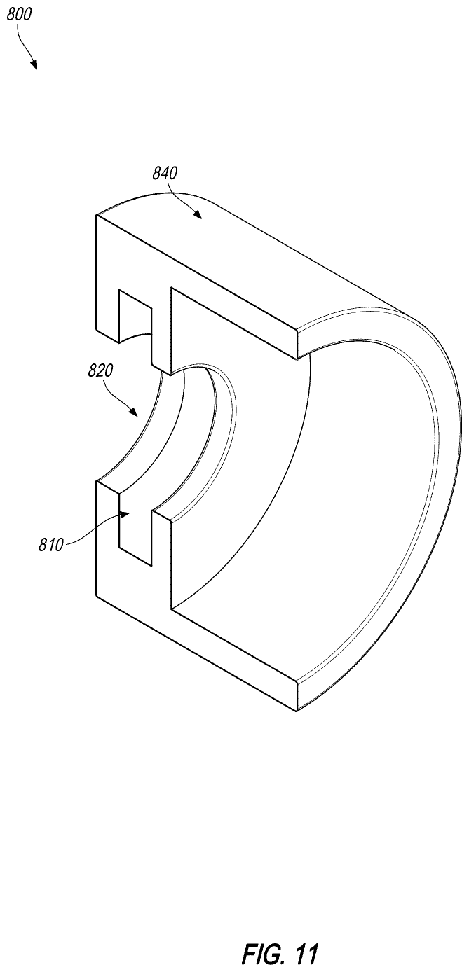

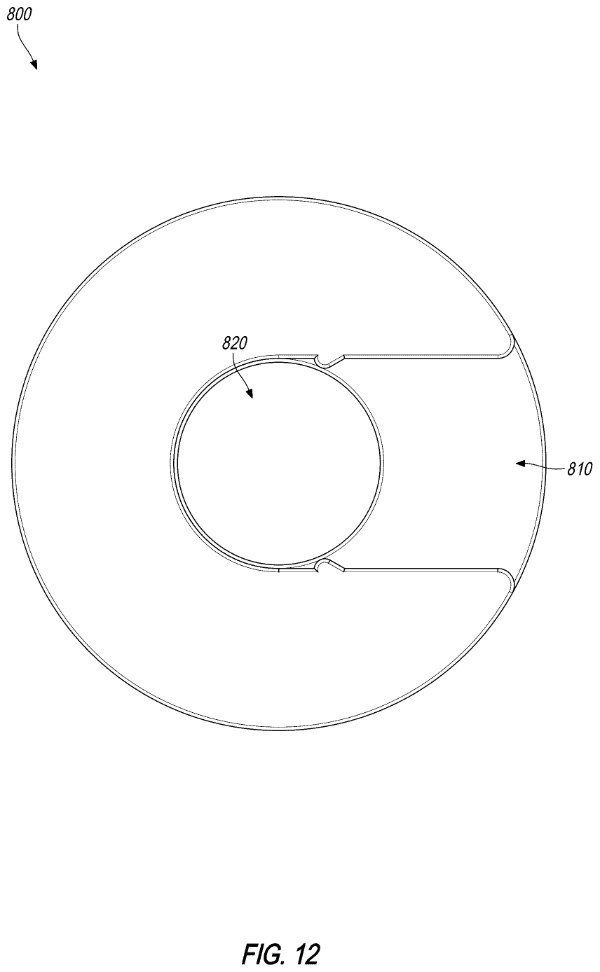

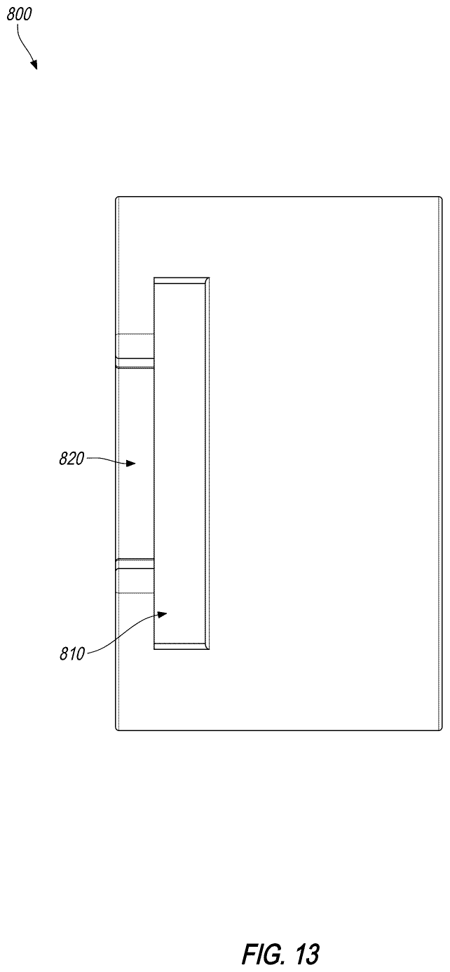

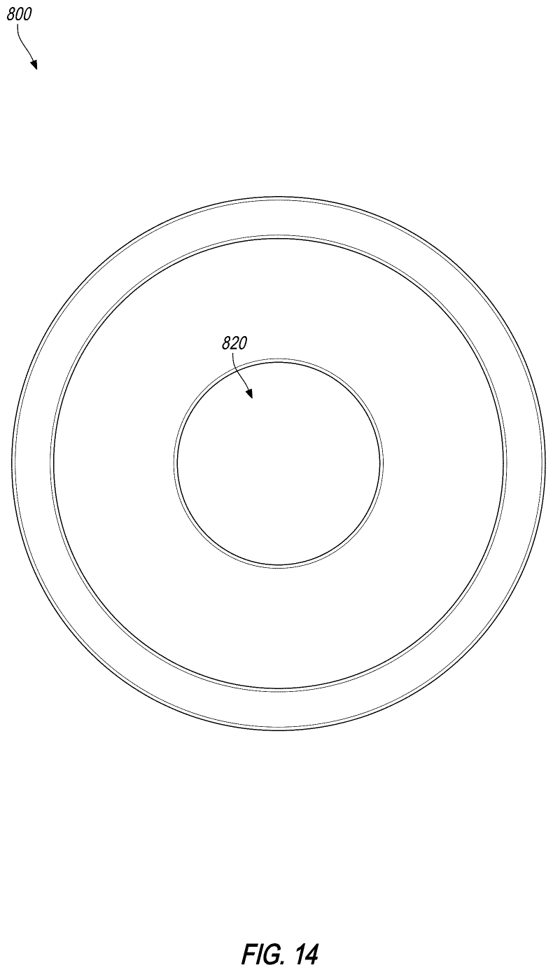

FIGS. 10 and 11 are back perspective and cutaway back perspective views of a shroud (800) for use with a syringe body (34) of an injection system (600) according to some embodiments. The shroud (800) defines side-opening slot (810) and a central opening (820). FIGS. 12, 13, and 14 are front, side, and back views of a shroud (800) for use with a syringe body (34) of an injection system (600) according to some embodiments. As shown in FIGS. 10 and 11, the shroud (800) has a circumferential surface (840) configured to interact/register with a corresponding component of a two-part injector having an external housing (see FIG. 17).

The sizes of the circumferential surface (840) and the site-opening slot (810) provide ranges of tolerance for various dimensions of the syringe body (34) and the syringe flange (38). In some embodiments, the range of tolerance for the length of the syringe body (34) is −1.5 mm to +1 mm. in some embodiments, the range of tolerance for the diameter of the syringe body (34) is −0.5 mm to +0.5 mm. in some embodiments, the range of tolerance for the diameter of the syringe flange (38) is −0.75 mm to +0.75 mm. The shroud (800) is configured to allow syringe bodies (34) and syringe flange's (38) with lengths and diameters in these ranges of tolerance to interact/register with a corresponding component of a two-part injector having an external housing (see FIG. 17). In some embodiments, the range of tolerance of the shroud (800) for the syringe body (34) includes a length tolerance configured to maintain a predetermined depth of the distal needle member (48) to be inserted into a patient by an autoinjector (1100; see FIG. 17- and 29).

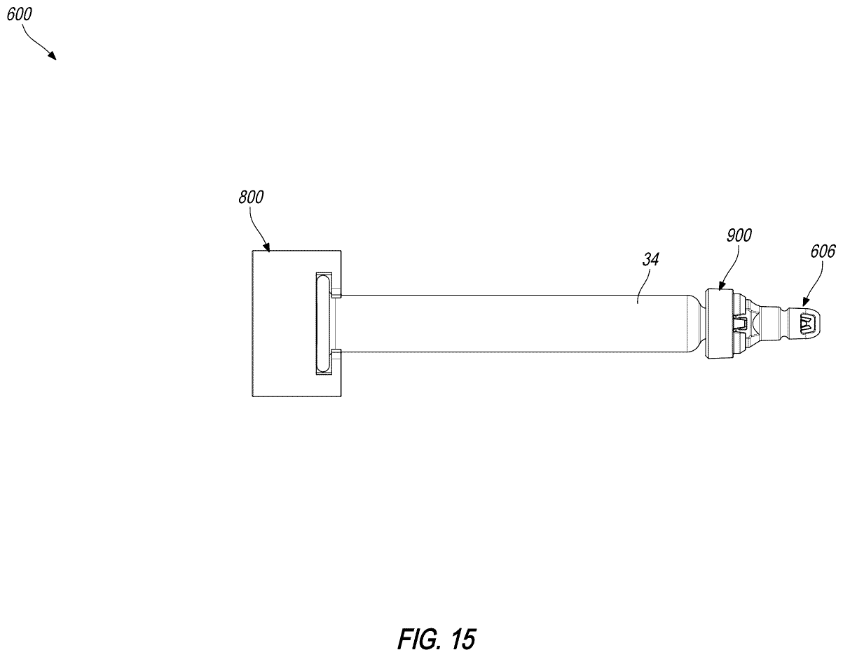

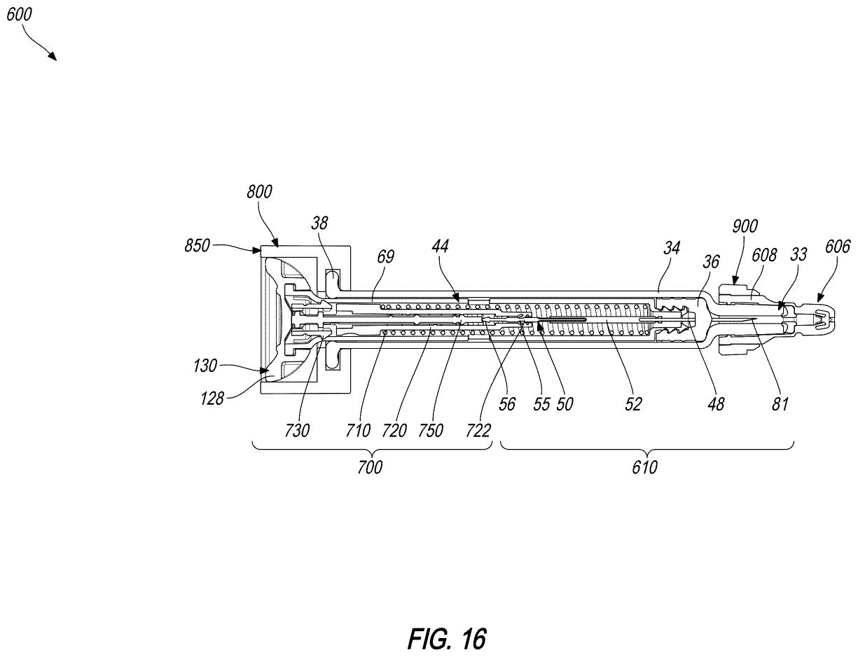

FIGS. 15 and 16 are perspective and longitudinal cross-sectional views of a safe injection system (600) in a safe configuration after injection according to some embodiments. In FIGS. 15 and 16, distally directed force has been applied to the stopper member (36) through the proximal interface (128) and the plunger member (44) thereby moving the stopper member (36) to the distal end (33) of the syringe body (34) and ejecting a liquid from the syringe chamber (40). The radially expanded member (56) of the needle proximal member (50) has moved proximally through the stopper member (36) and past the needle latches (722) in the needle catch (720). The radially expanded member (56) has also moved the trigger (750) proximally to allow the energy-storage member latches (730) to rotate inward away from the plunger body (69) to transform the energy-storage member latches (730) from a latched state to an unlatched state. This transformation of the energy-storage member latches (730) allows the energy storage member (710) to transform from an energized state/compressed to an unenergized/expanded state. The proximally expanding energy-storage member (710) moves the needle catch (720) and its needle latch arms (722) proximally such that the needle latch arms (722) engage the radially expanded member (56) of the needle proximal member (50) to pull the needle assembly (610) in a proximal direction.

The released energy storage member (710) fully expands proximally, thereby moving the needle catch (720) proximally in the plunger interior (70) and retracting the needle assembly (610) partially into the plunger interior (70). After the needle assembly (610) is retracted, the sharp needle distal end (81) is disposed in the needle hub (608) rendering the system (600) safe for disposal while minimizing unintended needle sticks. Disposing of a safe injection system (600) after injection and needle retraction may include removing the safe injection system (600) from an autoinjector, thereby allowing the autoinjector to be reused in some embodiments.

In the safe configuration depicted in FIGS. 15 and 16, the proximal interface (128) is disposed in an interior of the shroud (800) with a proximal surface (130) of the proximal interface (128) distal of a proximal surface (850) of the shroud (800). The proximal interface (128) has an outer diameter less than an inner diameter of the shroud (800) to enable the proximal interface (128) to be disposed in the interior of the shroud (800). The proximal interface (128) being disposed in the interior of the shroud (800) renders the post injection safe injection system (600) “tamperproof,” in that it is very difficult for a user to manually remove the plunger member (44) and the needle assembly (610) from the system (600). This minimizes the risk of unintended needle sticks.

FIG. 17 is a schematic view of a safe injection system (600) disposed in a two-part injector (1000) having an external housing (1100) in a ready-to-use configuration according to some embodiments. The safe injection system (600) is in a ready-to-use configuration such as that depicted in FIG. 7. The external housing (1100) includes proximal and distal registration surfaces (1110, 1120) configured to interact/register with the shroud (800) and the compression ring (900) of the safe injection system (600) respectively to secure the safe injection system (600) in the external housing (1100). The proximal and distal registration surfaces (1110, 1120) may face proximally, distally, and/or radially inward. The external housing (1100) also includes a manipulator (1130) configured to, automatically or manually, apply a distally directed force to the proximal interface (128) to advance the plunger member (44) and the stopper member (36 attached thereto distally in the syringe chamber (40).

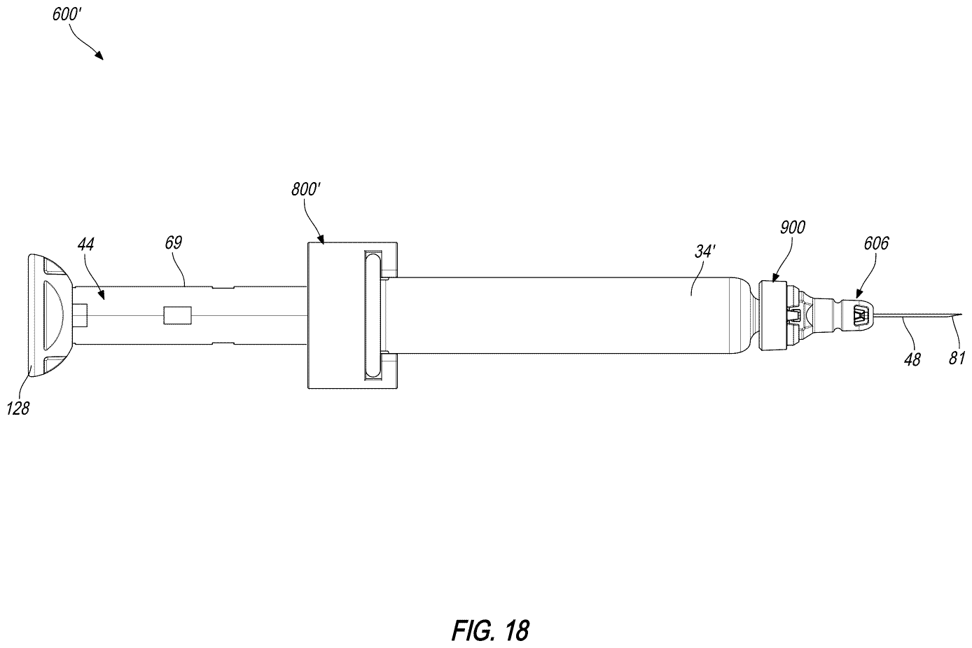

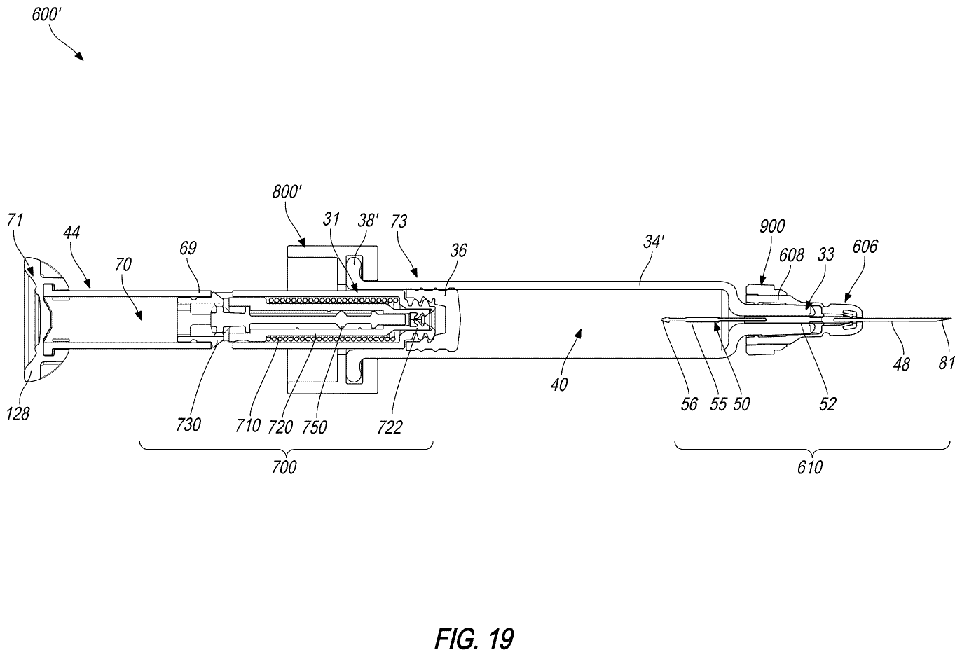

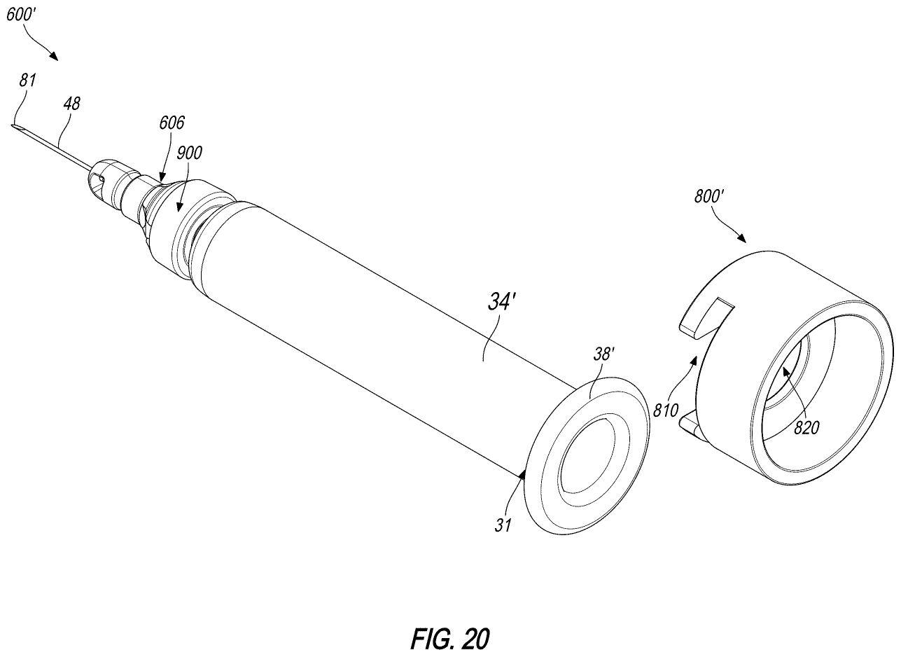

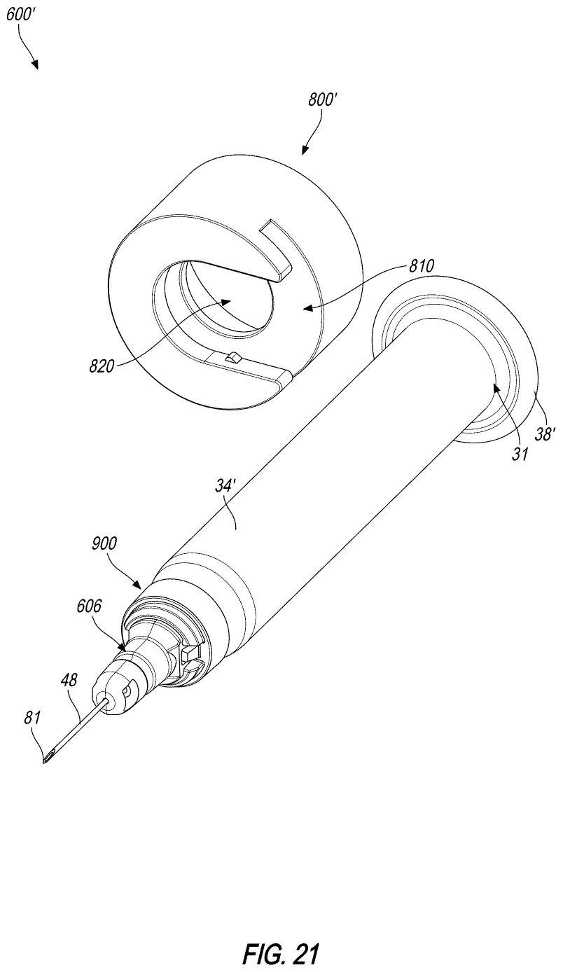

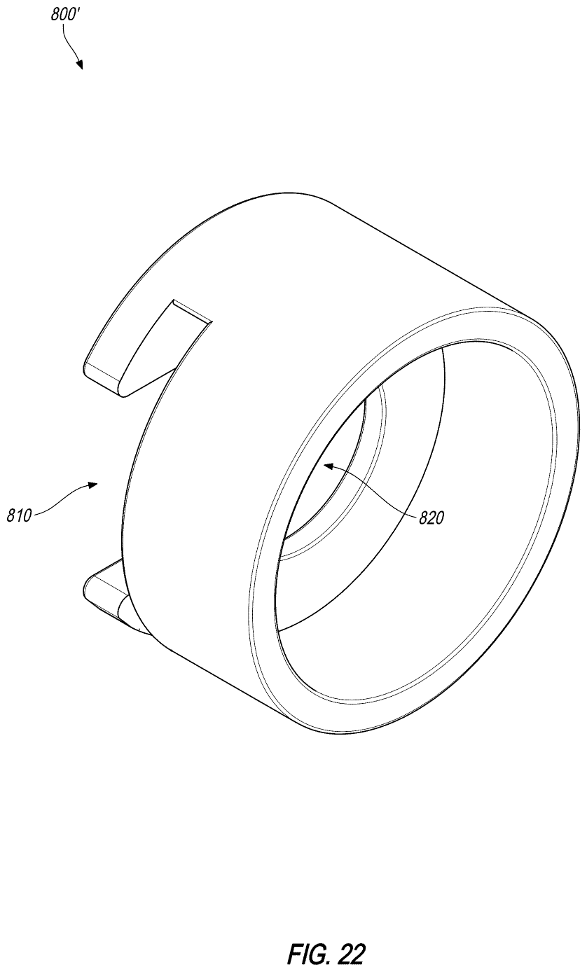

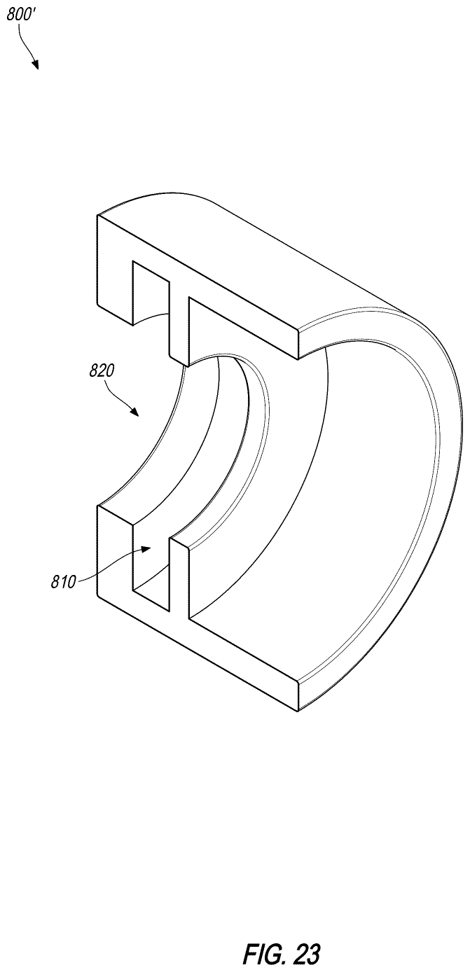

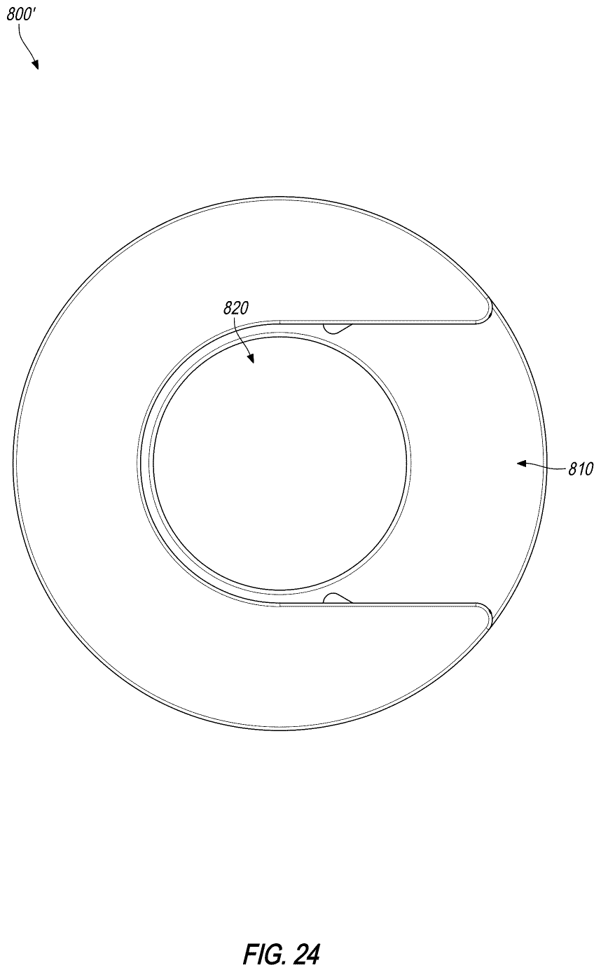

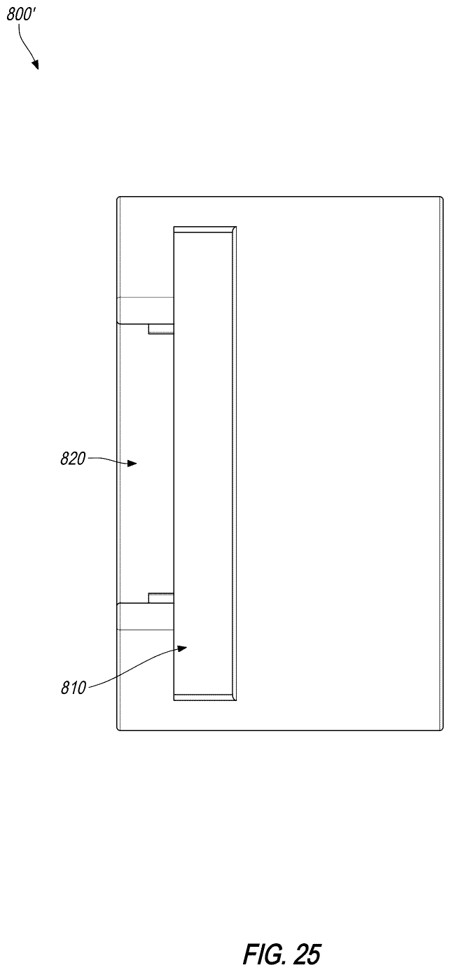

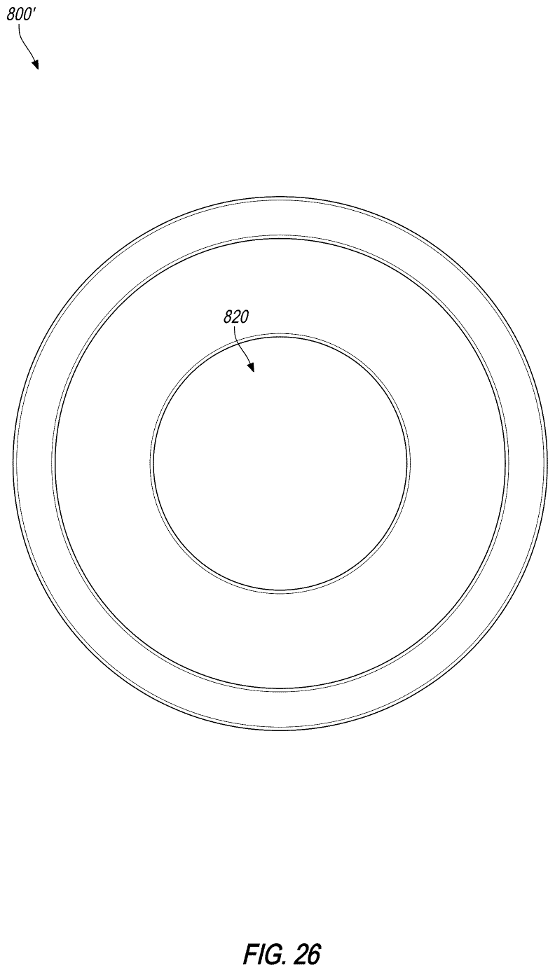

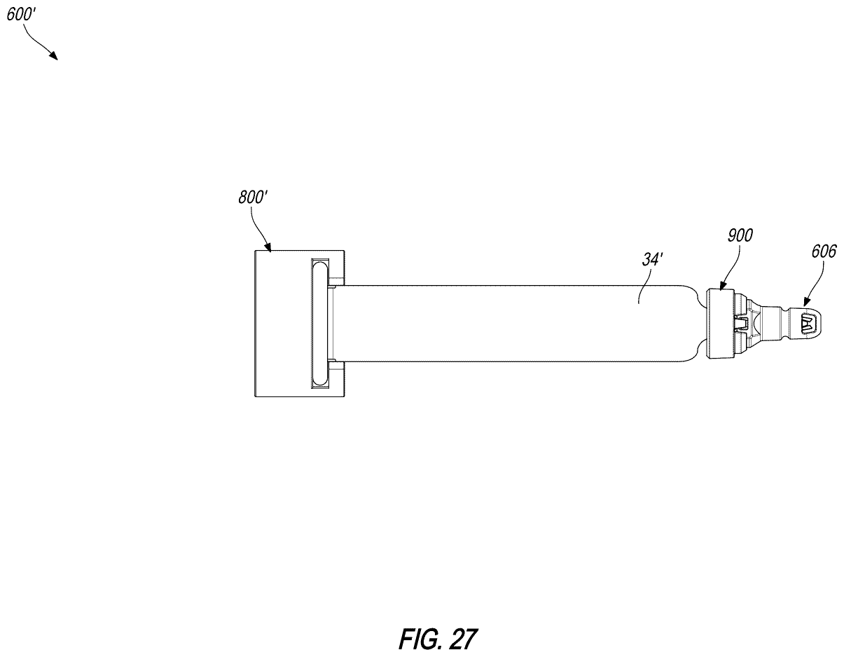

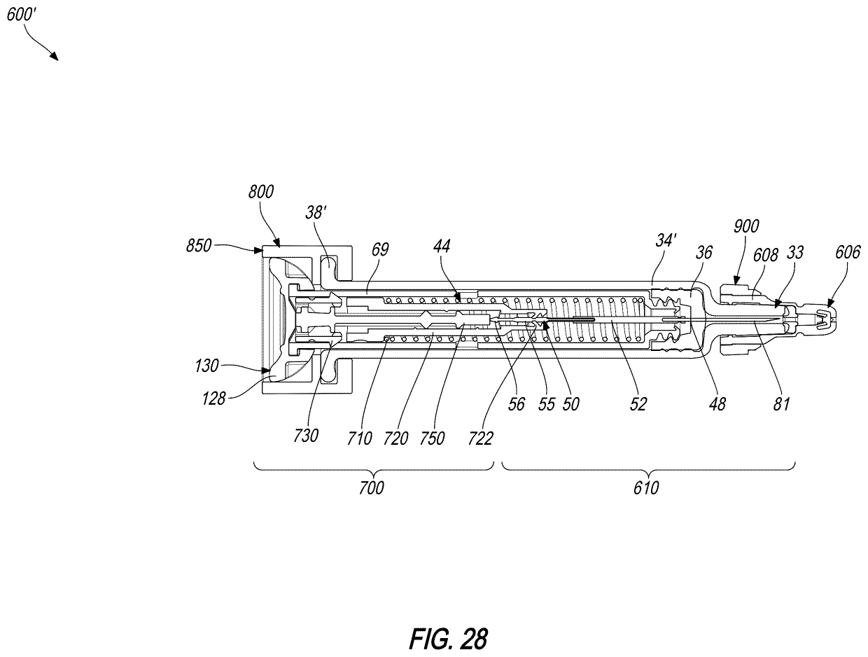

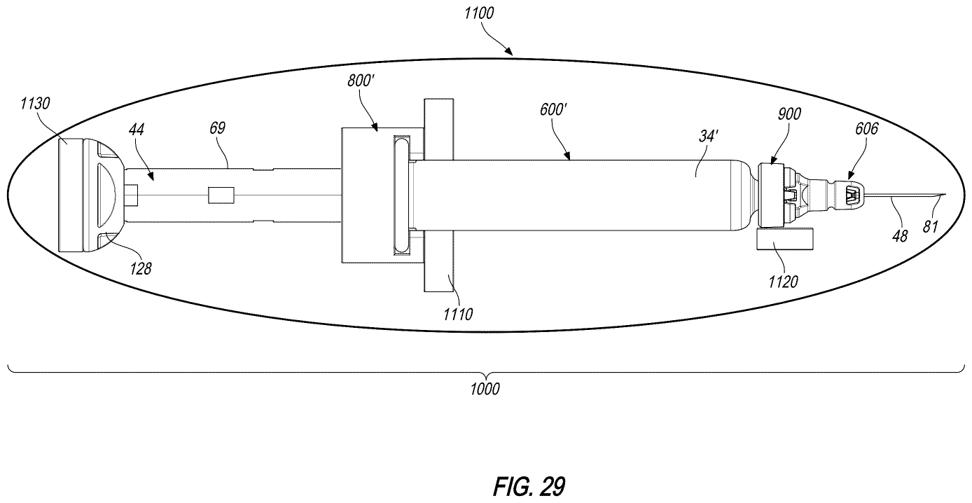

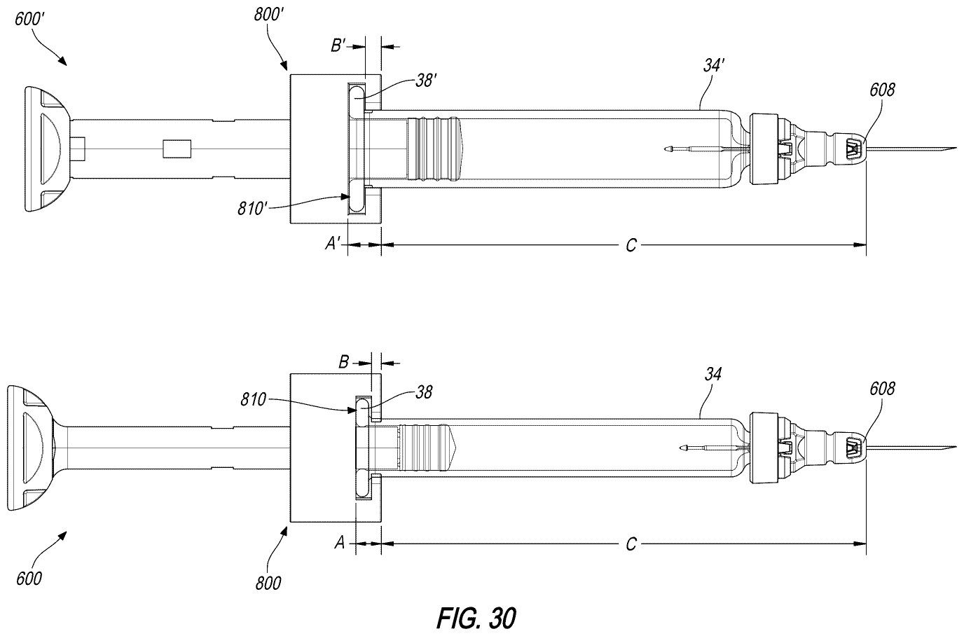

FIGS. 18 to 28 are various views of a safe injection system (600′) and components thereof according to some embodiments. FIG. 29 is a schematic view of a safe injection system (600′) disposed in a two-part injector (1000) having an external housing (1100) in a ready-to-use configuration according to some embodiments. The safe injection system (600′) is almost identical to the safe injection system (600) depicted in FIGS. 7 to 17. The differences derive from the fact that the syringe body (34′) of the safe injection system (600′) has at least one different dimension compared to the syringe body (34) of the safe injection system (600). The syringe body (34′) is configured to be prefilled with a volume of medicine between approximately 0.2 ml to approximately 10 ml of liquid. The illustrative embodiments shown depict syringe bodies sized to deliver volumes of 1 ml, 2.25 ml, and/or 3 ml of medicine. The shroud (800′) in the safe injection system (600′) is configured to couple to the syringe flange (38′) while maintaining a distance (C) between a distal end of the shroud (800′) and a distal end of the needle hub (608) substantially identical to the distance (C) between a distal end of the shroud (800) and a distal end of the needle hub (608) of the safe injection system (600) as shown in FIG. 30. In the safe injection systems (600, 600′) depicted in FIG. 30, the distances (C) between the respective distal ends of the shrouds (800, 800′) and the distal ends of the needle hubs (608) are identical (i.e., 2.70 in). The distance (A) between a proximal end of the side-opening slot (810) and a distal end of the shroud (800) is 0.140 ins. The distance (A′) between a proximal end of the side-opening slot (810′) and a distal end of the shroud (800′) is 0.180 ins. The distance (B) between a distal end of the side-opening slot (810) and a distal end of the shroud (800) is 0.089 ins. The distance (B′) between a distal end of the side-opening slot (810′) and a distal end of the shroud (800′) is 0.059 ins. Distances (A), (A′), (B), and (B′) configured the shrouds (800, 800′) such that distances (C) and (C′) are identical. As such, the safe injection systems (600, 600′) are configured for use in identical external housings (1100) having identical proximal registration surfaces (1110). The shrouds (800, 800′) are configured to dispose the distal end of the needle hub (608) at the same location in the external housing (1100), i.e., adjacent the distal registration surface (1120) (see FIGS. 17 and 29).

Other components of the safe injection system (600′) are identical or similar to the corresponding parts of the safe injection system (600). Therefore, these parts of the safe injection system (600′) have reference numbers identical or similar to the corresponding parts of the safe injection system (600).

While the embodiments described above include single chamber safe injection systems, the scope of the claims also include multiple chamber injection systems. For multiple chamber safety injection systems, two or more stopper members are inserted into an injection system body (e.g., syringe body, cartridge body, etc.) to define a corresponding number of chambers.

While the safe injection systems depicted and described herein include syringe bodies with staked needles, the safe injection systems described herein can include syringe bodies with Luer connectors, etc.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. An injection system, comprising:
a syringe body, comprising
a syringe body proximal end,
a syringe body distal end,
a syringe body interior therebetween, and
a syringe flange at the syringe body proximal end;
a needle hub assembly coupled to the syringe body at the syringe body distal end, and comprising
a needle hub,
a needle removably coupled to the needle hub and having a sharp needle distal end and a needle proximal end feature, and
a compression ring disposed around a proximal end of the needle hub and configured to couple the needle hub to the syringe body distal end;
a stopper member movably disposed in the syringe interior;
a plunger member having a plunger member proximal end and a plunger member distal end, and coupled to the stopper member at the plunger member distal end,
wherein the plunger member is configured to insert the stopper member distally in the syringe interior relative to the syringe body and the needle hub; and
a shroud coupled to the syringe flange, wherein the shroud is configured to couple to the syringe flange within one or more ranges of tolerance for the syringe body.

2. The injection system of claim 1, wherein the one or more ranges of tolerance comprises a range of tolerance for a length of the syringe body.

3. The injection system of claim 2, wherein the range of tolerance for the length of the syringe body is −1.5 mm to +1 mm.

4. The injection system of claim 1, wherein the one or more ranges of tolerance comprises a range of tolerance for a diameter of the syringe body.

5. The injection system of claim 4, wherein the range of tolerance for the diameter of the syringe body is −0.5 mm to +0.5 mm.

6. The injection system of claim 1, wherein the one or more ranges of tolerance comprises a range of tolerance for a diameter of the syringe flange.

7. The injection system of claim 6, wherein the range of tolerance for the diameter of the syringe flange is −0.75 mm to +0.75 mm.

8. The injection system of claim 1, wherein the compression ring is configured to function as a registration flange for an autoinjector.

9. The injection system of claim 1, wherein the shroud is configured to function as a registration flange for an autoinjector.

10. The injection system of claim 1, wherein the syringe body interior is configured to hold 1 ml of liquid.

11. The injection system of claim 1, wherein the syringe body interior is configured to hold 2.5 ml of liquid.

12. The injection system of claim 1, further comprising a needle retraction system configured to retract the sharp needle distal end into the needle hub to render the injection system safe.

13. An injection system, comprising:
an autoinjector, comprising
a housing;
a syringe, comprising
a syringe body, comprising
a syringe body proximal end,
a syringe body distal end,
a syringe body interior therebetween, and
a syringe flange at the syringe body proximal end;
a needle hub assembly coupled to the syringe body at the syringe body distal end, and comprising
a needle hub,
a needle removably coupled to the needle hub and having a sharp needle distal end and a needle proximal end feature, and
a compression ring disposed around a proximal end of the needle hub and configured to couple the needle hub to the syringe body distal end;
a stopper member movably disposed in the syringe interior;
a plunger member having a plunger member proximal end and a plunger member distal end, and coupled to the stopper member at the plunger member distal end,
wherein the plunger member is configured to insert the stopper member distally in the syringe interior relative to the syringe body and the needle hub; and
a shroud coupled to the syringe flange, wherein the shroud is configured to couple to the syringe flange within one or more ranges of tolerance for the syringe body.

14. The injection system of claim 13, wherein the one or more ranges of tolerance comprises a range of tolerance for a length of the syringe body.

15. The injection system of claim 14, wherein the range of tolerance for the length of the syringe body is −1.5 mm to +1 mm.

16. The injection system of claim 13, wherein the one or more ranges of tolerance comprises a range of tolerance for a diameter of the syringe body.

17. The injection system of claim 16, wherein the range of tolerance for the diameter of the syringe body is −0.5 mm to +0.5 mm.

18. The injection system of claim 13, wherein the one or more ranges of tolerance comprises a range of tolerance for a diameter of the syringe flange.

19. The injection system of claim 18, wherein the range of tolerance for the diameter of the syringe flange is −0.75 mm to +0.75 mm.

20. The injection system of claim 13, wherein the compression ring is configured to function as a registration flange for an autoinjector.

21. The injection system of claim 20, wherein the autoinjector comprises a distal syringe holder operatively coupled to the compression ring.

22. The injection system of claim 13, wherein the shroud is configured to function as a registration flange for an autoinjector.

23. The injection system of claim 22, wherein the autoinjector comprises a proximal syringe holder operatively coupled to the compression ring.

24. The injection system of claim 13, wherein the syringe body interior is configured to hold 1 ml of liquid.

25. The injection system of claim 13, wherein the syringe body interior is configured to hold 2.5 ml of liquid.

26. The injection system of claim 13, further comprising a needle retraction system configured to retract the sharp needle distal end into the needle hub to render the syringe safe.

27. The injection system of claim 13, wherein the one or more ranges of tolerance of the shroud for the syringe body comprises a length tolerance configured to maintain a predetermined depth of the needle to be inserted into a patient by the autoinjector.

28. The injection system of claim 13, wherein the plunger member comprises a proximal interface as the plunger member proximal end, and
wherein in a safe configuration after injection, the proximal interface is disposed in an interior of the shroud with a proximal surface of the proximal interface disposed distal of a proximal end surface of the shroud.
